(19) United States
(12) Patent Application Publication
(10) Pub. No.: US 2024/0341616 A1
(43) Pub. Date: Oct. 17, 2024

(54) ESTIMATING DIAGNOSTIC QUALITY OF IMAGE FROM MR SIGNALS

(71) Applicant: Children’s Medical Center Corporation, Boston, MA (US)

(72) Inventors: Simon K. Warfield, Boston, MA (US); Tess E. Wallace, Boston, MA (US); Onur Afacan, Boston, MA (US)

(73) Assignee: Children’s Medical Center Corporation, Boston, MA (US)

(21) Appl. No.: 18/291,540

(22) PCT Filed: Jul. 22, 2022
(86) PCT No.: PCT/US22/74044

§ 371 Date: Jan. 23, 2024

Related U.S. Application Data
(60) Provisional application No. 63/225,251, filed on Jul. 23, 2021.

(51) Int. Cl. A61B 5/055; A61B 5/00

(52) U.S. Cl. CPC A61B 5/055; A61B 5/7221

(57) ABSTRACT

Described herein are techniques to determine whether an image that would result from a magnetic resonance imaging (MRI) scan would satisfy quality metrics, such as by being of sufficient quality to permit a diagnostic read of the image. The determination may be based on signals emitted and captured by an MRI system during the MRI scan, permitting an automated determination of quality of an image that would result from an MRI scan. Some such techniques may be performed during the MRI scan, before the completion of the MRI scan, or after the MRI scan before or after magnetic resonance data (MR data) resulting from the MRI scan has been processed to generate an image from the MR data.

### CROSS REFERENCE TO RELATED APPLICATIONS

The present application claims the benefit under 35 U.S.C. § 119 (e) of U.S. Provisional Patent Application No. 63/225,251, filed Jul. 23, 2021, and titled “Estimating Diagnostic Quality of Image From MR Signals,” the entire contents of which are incorporated herein by reference.

### BACKGROUND

Magnetic resonance imaging (MRI) is a non-invasive and versatile technique for imaging biological systems. Generally, MRI operates by detecting magnetic resonance (MR) signals, which are electromagnetic waves emitted by atoms in response to an applied electromagnetic field. The detected MR signals may then be used to generate images of tissues of a patient, usually internal to the patient and unable to be directly viewed without invasive surgery.

### SUMMARY

In one embodiment, there is a method, there is provided a method of evaluating at least one image that would result from a magnetic resonance image (MRI) scan, the method comprising: evaluating magnetic resonance (MR) signals captured during the MRI scan; determining a diagnostic quality of the at least one image that would result from the MRI scan, based at least in part on a result of evaluating the MR signals; and outputting an indication of the diagnostic quality of the at least one image.

In another embodiment, there is provided a system configured to evaluate a magnetic resonance image (MRI) scan, the system comprising at least one processor and at least one non-transitory computer-readable storage medium storing processor executable instructions that, when executed by the at least one processor, cause the at least one processor to perform a method comprising evaluating magnetic resonance (MR) signals captured during the MRI scan; determining a diagnostic quality of the at least one image that would result from the MRI scan, based at least in part on a result of evaluating the MR signals; and outputting an indication of the diagnostic quality of the at least one image.

In another embodiments, there is provided a magnetic resonance imaging (MRI) system configured to evaluate a magnetic resonance image (MRI) scan, comprising: an MRI scanner; at least one processor; and at least one non-transitory computer-readable storage medium storing executable instructions that, when executed by the at least one processor, cause the at least one processor to perform: evaluating magnetic resonance (MR) signals captured during the MRI scan; determining a diagnostic quality of the at least one image that would result from the MRI scan, based at least in part on a result of evaluating the MR signals; and outputting an indication of the diagnostic quality of the at least one image.

In another embodiment, there is provided a method for evaluating a magnetic resonance image (MRI) scan, the method comprising: estimating motion of a subject during an MRI scan based on measured magnetic resonance (MR) signals captured during the MRI scan; determining a diagnostic quality of the MRI scan based on the estimated motion, and outputting an indication of the diagnostic quality of the MRI scan.

In a further embodiment, there is provided a system configured to evaluate a magnetic resonance image (MRI) scan, the system comprising at least one processor and at least one non-transitory computer-readable storage medium storing processor executable instructions that, when executed by the at least one processor, cause the at least one processor to perform a method for evaluating a magnetic resonance image (MRI) scan, the method comprising: estimating motion of a subject during an MRI scan based on measured magnetic resonance (MR) signals captured during the MRI scan; determining a diagnostic quality of the MRI scan based on the estimated motion; and outputting an indication of the diagnostic quality of the MRI scan.

In another embodiment, there is provided a magnetic resonance imaging (MRI) system configured to evaluate a magnetic resonance image (MRI) scan. The MRI system comprises an MRI scanner, at least one processor, and at least one non-transitory computer-readable storage medium storing executable instructions that, when executed by the at least one processor, cause the at least one processor to perform a method for evaluating a magnetic resonance image (MRI) scan, the method comprising: estimating motion of a subject during an MRI scan based on measured magnetic resonance (MR) signals captured during the MRI scan; determining a diagnostic quality of the MRI scan based on the estimated motion; and outputting an indication of the diagnostic quality of the MRI scan.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic diagram of a magnetic resonance imaging (MRI) facility for evaluating an MRI scan, in accordance with some embodiments of the technology described herein.

FIG. 2 is a flowchart of an illustrative process 200 of evaluating an MRI scan, in accordance with some embodiments of the technology described herein.

FIG. 3 is a flowchart describing a process 300 for evaluating prediction of diagnostic image quality using free-induction decay (FID) navigator signals, in accordance with some embodiments of the technology described herein.

FIG. 4 is a series of example images acquired in pediatric patients, representative of different motion grades, as evaluated by a radiologist, according to some embodiments of the technology described herein.

FIG. 5A shows multi-channel FID navigator signal traces corresponding to images acquired with each motion grade, according to some embodiments of the technology described herein.

FIG. 5B shows the computation of the normalized mean absolute change FIDₙₐᵥ_{_{Δ}} from the multi-channel FID navigator data, and the corresponding integrated and partition-weighted integrated motion scores (shown in mm s⁻¹), according to some embodiments of the technology described herein.

FIG. 5C shows the computation of the cross-correlation coefficient FIDₙₐᵥ_{_{CCC}} from the multi-channel FID navigator data, and the corresponding integrated and partition-weighted integrated motion scores (shown in mm s⁻¹), according to some embodiments of the technology described herein.

FIG. 6A is a series of boxplots showing the distribution of integrates FID navigator metrics corresponding to each image grade, as evaluated by a radiologist, according to some embodiments of the technology described herein.

FIG. 6B is a series of boxplots showing the distribution of partition-weighted integrated FID navigator metrics corresponding to each image grade, as evaluated by a radiologist, according to some embodiments of the technology described herein.

FIG. 7A shows the receiver operating characteristic (ROC) curves and operating points for each integrated FID navigator metric showing sensitivity and specificity for detecting the difference in motion corruption between image grades 2 and 3 (severe-moderate motion artifacts), according to some embodiments of the technology described herein.

FIG. 7B shows the receiver operating characteristic (ROC) curves and operating points for each integrated FID navigator metric showing sensitivity and specificity for detecting the difference in motion corruption between image grades 3 and 4 (moderate-mild motion artifacts), according to some embodiments of the technology described herein.

FIG. 8A is a histogram showing the proportion of MRI scans, of image grades 4 and 5, correctly flagged as diagnostic or non-diagnostic using thresholds from the ROC analysis, according to some embodiments of the technology described herein.

FIG. 8B is a histogram showing the proportion of MRI scans, of image grade 3, correctly flagged as diagnostic or non-diagnostic using thresholds from the ROC analysis, according to some embodiments of the technology described herein.

FIG. 8C is a histogram showing the proportion of MRI scans, of image grades 1 and 2, correctly flagged as diagnostic or non-diagnostic using thresholds from the ROC analysis, according to some embodiments of the technology described herein.

FIG. 9 is a schematic diagram of an illustrative computing device with which aspects described herein may be implemented.

### DETAILED DESCRIPTION

Described herein are techniques to determine whether an image that would result from a magnetic resonance imaging (MRI) scan (a magnetic resonance image, or “image”) would satisfy quality metrics, such as by being of sufficient quality to permit a diagnostic read of the image. The determination may be based on signals emitted and captured by an MRI system during the MRI scan, permitting an automated determination of quality of an image that would result from an MRI scan. Some such techniques may be performed during the MRI scan, before the completion of the MRI scan, or after the MRI scan before or after magnetic resonance data (MR data) resulting from the MRI scan has been processed to generate an image from the MR data.

Such techniques may be useful to imaging technologists by providing insights on whether motion of a patient be scanned is impacting or has impacted diagnostic quality of an image that would result from the MRI scan. These insights can be used to guide interventions for preventing further patient movements and for conducting additional scans according to imaging needs.

Rather than making the quality analysis based on a direct analysis of the image(s) that have resulted from processing of MR data captured during an MRI scan, some techniques described herein include analyzing signals captured during the MRI scan to determine the diagnostic quality of such images, including in some embodiments prior to generation of such an image and/or prior to completion of an MRI scan. More particularly, in some embodiments, MR signals that are captured during acquisition of data during an MRI scan of a subject (e.g., a patient) are analyzed to determine whether the subject moved during the scan. The MR signals may, in some cases, be analyzed to generate a quantitative metric of subject motion, which may then be evaluated to determine quality of an image that would result from the scan. For example, the metric may be compared to a threshold and, if the threshold is met (e.g., exceeded), this may be an indication that the subject motion was substantial enough to impede diagnostic image capture and/or that the image will not meet quality metrics. In other embodiments, the MR signals may be analyzed to generate a quantitative or qualitative conclusion with respect to quality metrics of an image, such as whether a resulting image would be of diagnostic quality, such as using a classifier technique or other analysis technique. Embodiments are not limited to any one particular type of signal processing technique.

In some cases, the MR signals that are analyzed to determine motion of a subject and/or estimate quality of an image that has or will result from an MRI scan, are or include MR signals captured during the MRI scan and during acquisition of data for that MRI scan, which in such embodiments may eliminate or mitigate a need to stop or pause an MRI scan to perform a separate, supplemental determination of motion or quality. The MR signals may be captured by receiving coils during the MRI scan and acquisition of signals may be part of a sequence for the MRI scan. Additionally or alternatively, in some cases, acquiring the MR signals that are to be analyzed to determine subject movement may include repeatedly acquiring the same point or region of k-space, such as by repeatedly acquiring a central k-space signal. A portion of a sequence that repeatedly samples a point or region of k-space and that may be used in some embodiments for acquiring MR signals for the analysis described herein may include radial spoke trajectory, spiral trajectory, Cartesian trajectory, or other trajectories. In some cases, it may be advantageous for MR signals captured during an MR scan, that are to be analyzed to determine subject motion and/or image quality, to be captured without gradient encoding. This may be the case, for example, where a point or region near a center of k-space is to be sampled repeatedly, as signals acquired near the center of k-space may have a higher signal-to-noise ratio. Other MR signals may be acquired without gradient encoding and analyzed as described herein, though, as embodiments are not limited in this respect.

In some embodiments, analyzing MR signals captured during an MRI scan to determine motion of a subject (e.g., patient) and/or image quality may include analyzing navigator signals captured during an MRI scan. The inventors have recognized and appreciated that, in at least some cases, the emission and capture of navigator signals can involve suspension of MR data acquisition, which can result in overall reduced acquisition efficiency. As such, described herein are some embodiments that include techniques for evaluating free-induction decay (FID) navigator signals that are captured by receiver coils without any spatial gradient encoding. Using such FID navigator signals in this way may enable faster acquisition with less impact on the magnetization or overall scan time. In some embodiments, the results of evaluating the FID navigator signals are used to determine a diagnostic quality of the image(s) that would result from the MRI scan. For example, in some embodiments, one or more complex signals from multiple channels may be extracted from one or more FID navigator signals captured during an MRI scan, and based on the complex signal(s) a quantitative motion metric may be determined and compared to one or more thresholds. Based on a result of the comparison, a determination of quality of an image that would or has resulted from processing of the signals from the MRI scan may be made.

As another example, in some embodiments, analyzing MR signals captured during an MRI scan to determine subject motion and/or image quality may include analyzing radial spoke signals. The central point of a radial spoke may be acquired during an MRI scan without any gradient encoding, and in such a situation may be viewed as a self-encoded FID navigator signal. Such a central point signal may thus be analyzed in a manner similar to analysis of FID navigator signals discussed above. Other signal trajectories may also include repeatedly acquiring a point or region of k-space, in some cases without gradient encoding, such as some spiral trajectories and others. Signals resulting from radial, spiral, and other trajectories may thus in some cases be viewable as self-encoded FID navigator signals. Analysis of signals that repeatedly acquire a point or region of k-space and/or that are captured without gradient encoding, may be analyzed to determine patient motion and/or image quality. For example, in some embodiments, one or more complex signals from multiple channels may be extracted from one or more MR signals captured during an MRI scan, and based on the complex signal(s) a quantitative motion metric may be determined and compared to one or more thresholds. Based on a result of the comparison, a determination of quality of an image that would or has resulted from processing of the signals from the MRI scan may be made. In embodiments in which different sequences and/or trajectories are used and in which results of signal analyses are compared to thresholds, different thresholds may be determined and used for different sequences/trajectories, for the purposes of determining motion and/or quality.

In some embodiments, an indication of diagnostic quality of an image that would result may be output to the imaging technologist. For example, the output may include an indication of the diagnostic quality. The indication may be qualitative or quantitative, and may be a binary indication (non-diagnostic or diagnostic) or may indicate a quality degree ranging from non-diagnostic to fully diagnostic, as embodiments are not limited in this respect.

An approach of evaluating MR signals (e.g., FID navigator signals) and determining diagnostic quality of an image based on the results of the evaluation of the MR signals may offer distinct advantages and increased accuracy over conventional approaches, as described below.

MRI is a non-invasive, versatile technique for studying physiology and pathology of a body, such as a human body or other animal body. MRI exploits the nuclear magnetic resonance (NMR) phenomenon to distinguish different structures, phenomena or characteristics of a subject. For example, in biological subjects, MRI may be employed to distinguish between various tissues, organs, anatomical anomalies (e.g., tumors), and/or to image diffusion, blood flow, blood perfusion, etc. In general, MRI operates by manipulating spin characteristics of subject material. MRI techniques include aligning the spin characteristics of nuclei of the material being imaged using a generally homogeneous magnetic field and perturbing the magnetic field with a sequence of radio frequency (RF) pulses. To invoke the NMR phenomenon, one or more resonant coils may be provided proximate a subject positioned within the magnetic field. The RF coils are adapted to generate RF pulses, generally in the form of RF pulse sequences adapted for a particular MRI application, to excite the nuclei and cause the spin to precess about a different axis (e.g., about an axis in the direction of the applied RF pulses). When an RF pulse subsides, spins gradually realign with the magnetic field, releasing energy that can be measured to capture NMR data about the internal structure of the subject. Measurement of the energy of the signals can generated data indicative of nuclei excitation and relaxation. These measurements of nuclei excitation and relaxation are not meaningful to or susceptible to human manual interpretation. But complex digital signal processing techniques may be implemented by a device (e.g., through execution of software implementing the signal processing techniques) to generate, from variations within that nuclei excitation and relaxation data, information on different structures that were subjected to the MRI scan and that correspond to those variations, which in the context of biological subjects may be information about tissues or other anatomy of the subject. Such complex signal processing may be used to generate MR images of the subject, such as images of anatomy of a biological subject.

An MRI scan is performed by an imaging technologist operating an MRI system, and an image is obtained through processing of the MR data that is obtained by the MRI system during the MRI scan. The imaging technician does not read the images or make any diagnoses. Instead, the detailed images generated using MRI are interpreted by a radiologist, such as to help diagnose or monitor treatment for a variety of conditions. The radiologist may guide imagining technologists in performing MRI scans and generating images for the radiologist's evaluation, but the radiologist does not operate the MRI system and often is not present during the MRI scan. Instead, the radiologist may be located in another area (perhaps not even in the same building or geographic area) and may be reviewing many images resulting from many MRI scans to perform diagnoses.

Generating an image that has diagnostic utility may depend on the stillness of the subject (e.g., patient) being scanned. Motion of a patient during an MRI scan can result in image artifacts including ghosting, blurring, and signal dropout that can significantly degrade image quality. If the image quality is sufficiently degraded, it may not be possible for a radiologist to make a diagnosis using the image. If a radiologist determines that a resulting image is of insufficient quality, it may be necessary to repeat the MRI scan and, in some cases, sedate the patient during the repeated MRI scan to keep them still. On top of disadvantages of a repeated MRI scan in terms of time and resources for the hospital and patient, a repeated MRI scan may be even more challenging because (as discussed below) there may be a significant time lag between the first MRI scan and when the radiologist has determined that the image is not of sufficient quality. That significant time lag between a first time MRI scan and any decision that a second or subsequent MRI scan is needed can have a significant negative impact on the patient, the hospital, the imaging technologies, and others.

In an attempt to prevent motion from degrading image quality, conventionally, an imaging technologist may visually monitor and provide instructions to a patient during the MRI scan. However, this may not be sufficient for preventing patient movement altogether, especially in children and other uncooperative patient populations. Moreover, if the patient does move, the imaging technologist has no way of determining if that particular motion will or will not impact the diagnostic quality of the image that will result from the MRI scan. Interrupting and repeating an MRI scan that may have resulted in a diagnostically useful image is a waste of time and resources. Therefore, an imaging technologist must wait until the MRI scan is complete and the image is generated prior to any decisions be made by a radiologist. Thus, after acquiring the MRI scan, the resulting image is checked for artifacts by the radiologist. Even in a case that the image does include artifacts that impact diagnostic quality, an imaging technologist (who is trained to operate the MRI system) is not trained or qualified to identify those artifacts in the image or determine whether the image can still be used by a radiologist to make a diagnosis. Furthermore, the imaging technologist is not trained to select alternative strategies for addressing the imaging needs. Providing these skills to every imaging technologist would require a substantial investment in training and education, as well as a long period of practical experience. Instead, a radiologist evaluates the resulting image to determine if it is of diagnostic quality or if another MRI scan needs to be acquired. However, in the usual imaging workflow, a radiologist may not be immediately available to assess image quality, as they may be interpreting or managing other cases. This may incur delays of fifteen minutes or more, in addition to the time already taken for completing the initial MRI scan or the time needed for repeat MRI scans to be completed and evaluated. During that time, in many cases, a patient may have left the hospital or, even if in the hospital, may have been kept waiting for a time the patient may find unacceptable.

The inventors have recognized and appreciated that such challenges and inefficiencies may be mitigated by automated systems to evaluate the diagnostic quality of an image that would result from an MRI scan and providing an indication of the diagnostic quality to the imaging technologist who is conducting the MRI scan.

The inventors have accordingly developed systems and methods for determining the diagnostic quality of an image that would result from an MRI scan. In some embodiments, determining diagnostic quality includes evaluating FID navigator signals captured by receiver coils during the MRI scan. The inventors have realized that FID navigators are sensitive to motion due to the localized spatial sensitivities of receiver coils in a multi-channel receiver array. As a result, in some embodiments, FID navigator signals may be evaluated for motion of the patient during the MRI scan. The techniques include determining a diagnostic quality of an image that would result from the MRI scan, based on the results of the evaluation, and outputting an indication of the diagnostic quality to the imaging technologist.

Accordingly, some embodiments provide for a method of evaluating FID navigator signals to determine diagnostic quality of an image that would result from an MRI scan. The method includes determining one or more MR signal-based (e.g., FID navigator-based) metrics that represent MR signals captured by multiple receiver coils over multiple channels over time, and determining the diagnostic quality of an image that would result based on such MR signal-based metrics. In some embodiments, the MR signal-based metrics may be indicative of motion of a subject during an MRI scan Some embodiments provide for a method of determining a diagnostic quality, of the image that would result from the MRI scan, from the MR signals. The method may include comparing the results of evaluating MR signals to one or more thresholds to determine diagnostic quality. For example, the techniques may include comparing a determined MR signal-based metric to a threshold to determine diagnostic quality. In some embodiments, this may include determining a degree of motion and/or a degree or diagnostic quality. For example, the techniques may include classifying a resulting image (either before or after the image has been generated) as diagnostic, non-diagnostic, or on a spectrum between diagnostic and non-diagnostic.

Various examples of ways in which these techniques and systems can be implemented are described below. It should be appreciated, however, that embodiments are not limited to operating in accordance with these examples. Other embodiments are possible.

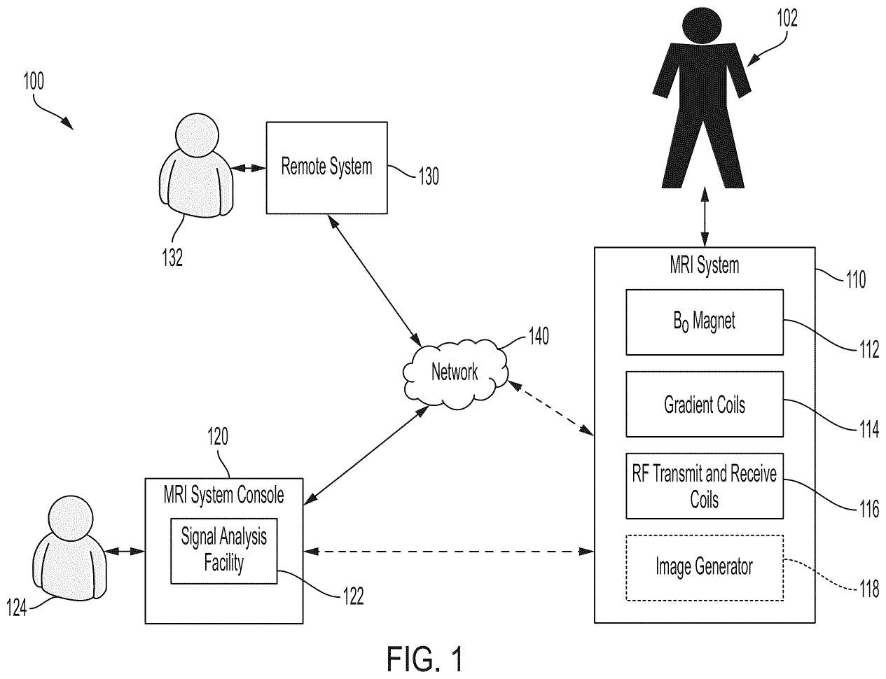

FIG. 1 is a block diagram of an example system 100 for evaluating MRI signals of an MRI scan to make estimations regarding an image that would result from the MRI scan, in accordance with some embodiments of the technology described herein. In the illustrative example of FIG. 1, system 100 includes an MRI system 110, an MRI system console 120, and a remote system 130. It should be appreciated that system 100 is illustrative and that a system may have one or more other components of any suitable type in addition to or instead of the components illustrated in FIG. 1. For example, there may be additional remote systems (e.g., two or more) present within a system.

As illustrated in FIG. 1, in some embodiments, one or more of the MRI system 110, the MRI system console 120, and the remote system 130 may be communicatively connected by a network 140. The network 140 may be or include one or more local and/or wide-area, wired, and/or wireless networks, including a local-area or wide-area enterprise network and/or the Internet. Accordingly, the network 140 may be, for example, a hard-wired network (e.g., a local area network within a healthcare facility), a wireless network (e.g., connected over Wi-Fi and/or cellular networks), a cloud-based computing network, or any combination thereof. For example, in some embodiments, the MRI system 110 and the MRI system console 120 may be located within the same healthcare facility and connected directly to each other or connected to each other via the network 140, while the remote system 130 may be located in a remote healthcare facility and connected to the MRI system 110 and/or the MRI system console 120 through the network 140.

In some embodiments, the MRI system 110 may be configured to perform MR imaging of anatomy of a patient 102. For example, the MRI system 110 may include a B0 magnet 112, gradient coils 114, and radio frequency (RF) transmit and receive coils 116 configured to act in concert to perform said MR imaging.

In some embodiments, B0 magnet 112 may be configured to generate the main static magnetic field, B0, during MR imaging. The B0 magnet 112 may be any suitable type of magnet that can generate a static magnetic field for MR imaging. For example, the B0 magnet 112 may include a superconducting magnet, an electromagnet, and/or a permanent magnet. In some embodiments, the B0 magnet 112 may be configured to generate a static magnetic field having a particular field strength. For example, the B0 magnet 112 may be a magnet that can generate a static magnetic field having a field strength of 1.5 T, or, in some embodiments, a field strength greater than or equal to 1.5 T and less than or equal to 3.0 T, or a field strength greater than or equal to 1.5 T and less than or equal to 7.0 T.

In some embodiments, gradient coils 114 may be arranged to provide one or more gradient magnetic fields. For example, gradient coils 114 may be arranged to provide gradient magnetic fields along three substantially orthogonal directions (e.g., x, y, and z). The gradient magnetic fields may be configured to, for example, provide spatial encoding of MR signals during MR imaging. Gradient coils 114 may comprise any suitable electromagnetic coils, including discrete wire windings coils and/or laminate panel coils.

In some embodiments, RF transmit and receive coils 116 may be configured to generate RF pulses to induce an oscillating magnetic field, B1, and/or to receive MR signals from nuclear spins within a target region of the imaged subject during MR imaging. The RF transmit coils may be configured to generate any suitable types of RF pulses useful for performing MR imaging. RF transmit and receive coils 116 may comprise any suitable RF coils, including volume coils and/or surface coils.

In some embodiments, the MRI system 110 may optionally include image generator 118. Image generator 118 may be configured to generate images based on MR data acquired by the MRI system 110 during MR imaging of the patient 102. For example, in some embodiments, image generator 118 may be configured to perform image reconstruction to generate images in the image domain based on MR data in the spatial frequency domain (e.g., MR data comprising data describing k-space).

As illustrated in FIG. 1, MRI facility 100 includes MRI system console 120 communicatively coupled to the MRI system 110. MRI system console 120 may be any suitable electronic device configured to send instructions and/or information to MRI system 110, to receive information from MRI system 110, and/or to process obtained MR data. In some embodiments, MRI system console 120 may be a fixed electronic device such as a desktop computer, a rack-mounted computer, or any other suitable fixed electronic device. Alternatively, MRI system console 120 may be a portable device such as a laptop computer, a smart phone, a tablet computer, or any other portable device that may be configured to send instructions and/or information to MRI system 110, to receive information from MRI system 110, and/or to process obtained MR data.

Some embodiments may include a signal analysis facility 122. Signal analysis facility 122 may be configured to analyze MR data obtained by MRI system 110 from an MR imaging procedure of patient 102. Signal analysis facility 122 may be configured to, for example, analyze the obtained MR data to make one or more determinations regarding the quality of an image that would be generated using the MR data, as described herein. Signal analysis facility 122 may be implemented as hardware, software, or any suitable combination of hardware and software, as aspects of the disclosure provided herein are not limited in this respect. As illustrated in FIG. 1, the signal analysis facility 122 may be implemented in the MRI system console 120, such as by being implemented in software (e.g., executable instructions) executed by one or more processors of the MRI system console 120. However, in other embodiments, the signal analysis facility 122 may be additionally or alternatively implemented at one or more other elements of the system 100 of FIG. 1. For example, the signal analysis facility 122 may be implemented at the MRI system 110 and/or the remote system 130 discussed below. In other embodiments, the signal analysis facility 122 may be implemented at or with another device, such as a device located remote from the system 100 and receiving data via the network 140.

MRI system console 120 may be accessed by MRI user 124 in order to control MRI system 110 and/or to process MR data obtained by MRI system 110. The MRI user 124 may be, for example, an imaging technologist. For example, MRI user 124 may implement an MR imaging process by inputting one or more instructions into MRI system console 120 (e.g., MRI user 124 may select an MR imaging process from among several options presented by MRI system console 120). Alternatively or additionally, in some embodiments, MRI user 124 may implement an MR data analysis procedure by inputting one or more instructions into MRI system console 120 (e.g., MRI user 124 may select MR data instances to be analyzed by MRI system console 120).

As illustrated in FIG. 1, MRI system console 120 also interacts with remote system 130 through network 140, in some embodiments. Remote system 130 may be any suitable electronic device configured to receive information (e.g., from MRI system 110 and/or MRI system console 120) and to display generated images for viewing. The remote system 130 may be remote from the MRI system 110 and MRI system console 120, such as by being located in a different room, wing, or building of a facility (e.g., a healthcare facility) than the MRI system 110, or being geographically remote from the system 110 and console 120, such as being located in another part of a city, another city, another state or country, etc. In some embodiments, remote system 130 may be a fixed electronic device such as a desktop computer, a rack-mounted computer, or any other suitable fixed electronic device. Alternatively, remote system 130 may be a portable device such as a laptop computer, a smart phone, a tablet computer, or any other portable device that may be configured to receive and view generated images and/or to send instructions and/or information to MRI system console 120.

In some embodiments, remote system 130 may receive information (e.g., MR data analysis results, generated images) from MRI system console 120 and/or MRI system 110 over the network 140. A remote user 132 (e.g., a radiologist or other clinician, such as the patient's medical clinician) may use remote system 130 to view the received information on remote system 130. For example, the remote user 132 may view generated images using remote system 130 after the MRI user 124 has completed MR data analysis using MRI system 110 and/or MRI system console 120.

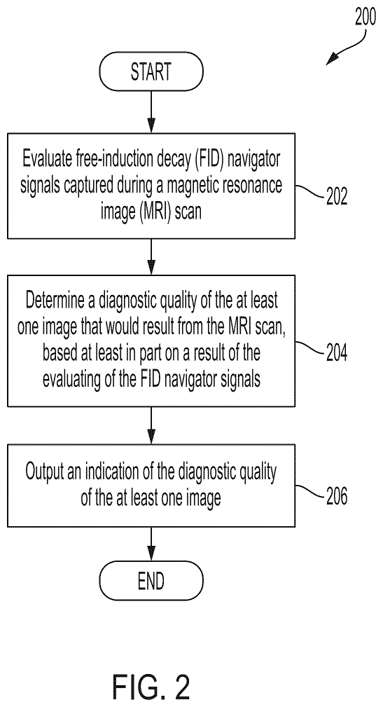

FIG. 2 is a flowchart of an illustrative process 200 for evaluating an MRI scan, in accordance with some embodiments of the technology described herein. Process 200 may be implemented by a signal analysis facility, such as the facility 122 of FIG. 1. As such, in some embodiments, the process 200 may be performed by a computing device configured to send instructions to an MRI system and/or to receive information from an MRI system (e.g., MRI system console 120 executing signal analysis facility 122 as described in connection with FIG. 1). As another example, in some embodiments, the process 200 may be performed by one or more devices/processors located remotely (e.g., as part of a cloud computing environment, as connected through a network) from the MRI system that obtained the input MR data.

Prior to the start of the process 200 of FIG. 2, the signal analysis facility may instruct the MRI system to acquire at least a portion of an MRI scan. In some embodiments, performing an MRI scan may include acquiring one or more MRI sequences. An MRI sequence may include a T1-weighted scan, a T2-weighted scan, a diffusion weighted scan, or any suitable MRI sequence, as aspects of the embodiments described herein are not limited in this respect. An MRI sequence may further include one or more embedded navigators, such as FID navigators. An FID navigator may be embedded in a sequence prior to an RF excitation pulse, following an RF excitation pulse, or at any suitable position in an MRI sequence, as aspects of the embodiments described herein are not limited in this respect. In some cases, the FID navigator may be embedded in the sequence following the RF excitation pulse, to measure the free induction decay following the pulse. The pulse that the FID navigator follows may be a pulse used for imaging, a pulse used for the purpose of acquiring an FID navigator signal, or other pulse at another suitable position in the MRI sequence. For example, the signal analysis facility may instruct the MRI system to perform an MRI scan with one or more FID-navigated sequences. In some embodiments, the instructions may specify parameters for acquiring an MRI sequence. For example, the parameters may specify a repetition time (TR), a time to echo (TE), a flip angle, receiver bandwidth, field-of-view (FOV), a spatial resolution, a generalized auto-calibrating partially parallel acquisition (GRAPPA) acceleration factor, a total acquisition time, or any other suitable parameter, as aspects of the technology described herein are not limited in this respect. In some embodiments, sequence parameters may be chosen to closely match those typically used in clinical studies.

In some embodiments, the signal analysis facility may specify acquisition settings for capturing FID navigator signals and/or the MRI sequence using the MRI system. For example, the signal analysis facility may specify an imaging trajectory, such as a Cartesian sampling trajectory with the center of the phase encoding direction acquired halfway through the MRI scan. As another example, the signal analysis facility may specify whether acquisitions are non-selective. As yet another example, the signal analysis facility may specify an acquisition plane, such as the sagittal, coronal, or transverse plane.

While examples are described herein, including in connection with this FIG. 2, of using FID navigator signals as an MR signal on which determinations of motion and/or image quality are based, it should be appreciated that embodiments are not limited to using FID navigator signals. As discussed above and in more detail below, other MR signals may be used, including MR signals captured from radial spoke trajectories, spiral trajectories, Cartesian trajectories, or other trajectories. Some such MR signals may repeatedly sample a same region or point in k-space, such as a central point in k-space. Some such MR signals may be acquired without gradient encoding.

After MRI sequence acquisition, the signal analysis facility may receive, from the MRI system, FID navigator signals captured during the MRI scan. In some embodiments, the FID navigator signals may have been captured by receiver coils, such as RF transmit and receive coils 116 of FIG. 1.

Process 200 begins at act 202 where the signal analysis facility evaluates the FID navigator signals captured during the MRI scan. In some embodiments, evaluating the captured FID navigator signals may include determining changes in the FID navigator signals over time. In some embodiments, changes in the FID navigator signals may be used to infer diagnostic quality of the image that would result from the MRI scan. For example, changes in the FID navigator signals may be indicative of patient motion during the scan.

Since FID navigator signals may be captured by multiple receiver coils over time, the signal analysis facility may determine one or more FID navigator-based metrics that represent changes in multiple such signals. For example, the signal analysis facility may combine FID navigator signals from N different channels into a single measurement, which may facilitate use of a threshold to determine diagnostic quality, as described herein including with respect to act 204 of process 200.

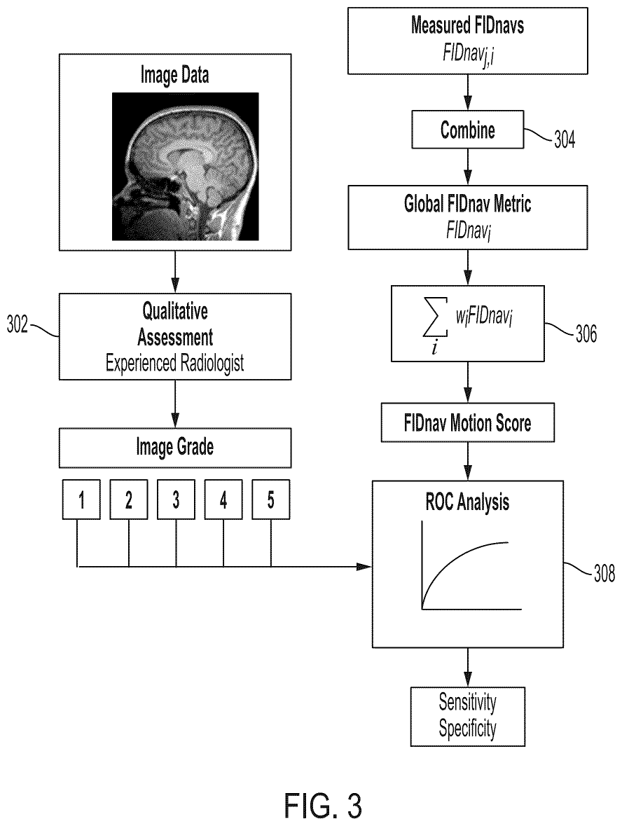

In some embodiments, evaluating the captured FID navigator signals (e.g., to determine FID navigator-based metrics) may first include combining coil measurements to determine a global FID navigator metric for a time point (or repetition time (TR)). In such embodiments, the “global” nature of the metric refers to the metric resulting from a combination of underlying signals, such as a combination of all signals for a channel, for a time (e.g., across multiple channels), or for an MRI scan (e.g., across multiple channels and times). To do so, according to some embodiments, the signal analysis facility may average FID navigator samples from readouts in one or more echo trains to yield a single complex navigator signal per channel for a TR. The complex navigator signals may then be combined to determine a global FID navigator metric. Embodiments are not limited to making this combination in any particular manner and may use suitable algorithms, such as the example algorithms described herein including with respect to FIG. 3.

In some embodiments, the global FID navigator metric represents a change in the FID navigator signals, captured over multiple channels, with respect to a previous TR. For example, the signal analysis facility may determine a normalized mean absolute change in the FID navigator signals over multiple channels between time points. In some embodiments, the signal analysis facility may determine a normalized mean absolute change over only some (e.g., two, three, four, five, etc.) of the channels. For example, the signal analysis facility may determine the normalized mean absolute change over the channels that experience the most change in the FID navigator signals between time points. In some embodiments, averaging over the most changing channels may reduce the effect of random signal fluctuations, while increasing motion-induced signal changes. As another example of computing a global FID navigator metric, the signal analysis facility may determine a cross correlation coefficient (CCC) between absolute FID navigator signal projection vectors between time points. In some embodiments, a change in CCC relative to the previous TR may imply that the load distribution of the coil elements has changed. Such a change in load distribution may be indicative of patient motion during the MRI scan. Thus, the change in CCC may be usable in some embodiments as an indicator of motion.

In some embodiments, the signal analysis facility may determine a global FID navigator metric for some, most, or all of the time points. For example, the signal analysis facility may discard the first few (e.g., one, two, three, four, five, etc.) time points to allow the signal to reach steady state, prior to computing a global FID navigator metric.

In some embodiments, after computing global FID navigator metrics for respective time points, the signal analysis facility may further compress the global FID navigator metrics into a single FID navigator motion score, which may represent the total impact of motion occurring during the MRI scan. The combination of global scores into a single score may be done in a variety of ways, as embodiments are not limited in this respect. As an example, determining the FID navigator motion score may include computing a numerical integration of the global FID navigator metric over a number of k-space lines in the acquisition. As another example, determining the FID navigator motion score may include computing a partition-weighted numerical integration of the global FID navigator metrics over a number of k-space lines in the acquisition. In some embodiments, the partition-weighted metric may reflect the fact that k-space energy and hence the impact of motion occurring at each encoding step may be non-uniformly distributed along the phase-encoding direction.

At act 204, based at least in part on a result of evaluating the FID navigator signals at act 202, the signal analysis facility determines a diagnostic quality of the at least one image that would result from the MRI scan. In some embodiments, determining diagnostic quality may include comparing the evaluation result to one or more thresholds. For example, this may include comparing the FID navigator motion score, indicative of the FID navigator signals captured over time, to a threshold. In some embodiments, a threshold may distinguish between different grades in diagnostic quality. For example, a threshold may distinguish between an image that may have at least some diagnostic value and an image that may not have any diagnostic value. Examples of determining thresholds for distinguishing between different grades in diagnostic quality are described herein including in conjunction with act 308 of FIG. 3.

At act 206, the signal analysis facility outputs an indication of the diagnostic quality of the at least one image. In some embodiments, the signal analysis facility may output an indication at any suitable time prior to or after completion of the MRI scan. For example, the signal analysis facility may output the indication during the MRI scan, prior to image generation. In some embodiments, outputting the indication may include displaying MR data, such as the metrics generated at act 204, through a user interface of the MRI system. Additionally or alternatively, outputting the indication may include providing an intervention recommendation. For example, when it is determined, at act 206, that the image that would result from the MRI scan is non-diagnostic, the signal analysis facility may recommend that the imaging technologist repeat the MRI scan. As another example, when it is determined, at act 206, that the image that would result from the MRI scan may not be fully diagnostic (e.g., diagnostic quality compromised, but some information may still be obtained), then the signal analysis facility may recommend that the imaging technologist instruct the patient to keep still.

Following the outputting of act 206, the process 200 ends. Following the process 200, a variety of actions may be taken. For example, in some cases in which the process 200 is performed prior to the end of the MRI scan or prior to start of or completion of image generation from MR data, the MRI scan may be completed or one or more images may be generated. Images generated from MR data may also be stored and/or communicated to a technologist or radiologist, alone or together with the determined indication of diagnostic quality, and the determined indication of diagnostic quality may be stored. If a decision is made to repeat the MRI scan, the scan may be repeated. In a case in which an MRI scan is repeated, the process 200 may also be repeated with signals captured during the repeated MRI scan.

While an example has been described in connection with MR signals that are FID navigator signals, it should be appreciated that embodiments are not so limited. As mentioned above, MR signals captured using a radial spoke trajectory may be used in some embodiments, and MR signals captured using a spiral trajectory may be used in other embodiments. Still other MR signals resulting from other trajectories may be used in other embodiments. In some such cases, the MR signals may have been captured by a sequence/trajectory that included repeatedly sampling one region or point of k-space, such as a center point. In some such cases, the MR signals may additionally or alternatively have been captured without using gradient encoding.

Radial acquisitions may in some MRI scanning contexts be an attractive alternative to Cartesian sampling for motion-robust imaging since the center of k-space may be repeatedly sampled and may even be densely sampled or oversampled in some such radial acquisitions. In radial encoding, k-space samples are acquired along radial spokes that are rotated around the center, which results in a “KOOSH Ball” sampling pattern. This may be realized in some MR scans by playing readout gradients in X, Y, and Z directions simultaneously and modulating the amplitudes according to:

$${G}_{x} = sin ( ϕ ) \cdot cos ( ψ )$$

$${G}_{y} = sin ( ϕ ) \cdot sin ( ψ )$$

$${G}_{z} = cos ( ψ )$$

where ϕ is the polar angle and ψ is the azimuthal angle, which together define the 3D radial trajectory. Different schematics may be used for the ordering of spokes, including known schematics such as the “golden angle” and “spiral phyllotaxis.” In some cases, the effects of motion may be averaged and artifacts manifest as local blurring rather than ghosting, due to the manner in which scanning lines are processed in some techniques, such as by being equally weighted or otherwise treated as equally “important” in processing. Radial spoke scans may in some contexts be more robust to under sampling, and radial projections may be ordered to provide spatially and temporally uniform k-space coverage to further minimize the effects of data inconsistencies due to motion.

Because in some radial spoke sequences, the spokes all share a common center point, the center point of k-space may be repeatedly sampled during a radial spoke acquisition. Such repeated acquisition of central k-space signal can provide an opportunity in some embodiments for detecting and estimating motion from the MR signals captured during the radial spoke sequence, particularly the signals resulting from the center of k-space.

In some embodiments that use a radial spoke sequence/trajectory, the MRI scanner may be configured and operated to acquire a central point of the radial spokes of the sequence without gradient encoding. In this case, analyzing the MR signals to determine motion and/or quality may include analyzing MR signals corresponding to the central point. The MR signals corresponding to the central point may be analyzed in a manner similar to the discussion above of FID navigator signals, as due to the lack of gradient encoding the central point signals may be viewable as a self-encoded signal, and thus treatable in analysis like what is described above in connection with FIG. 2, and in more detail below in connection with FIG. 3, as a self-encoded FID navigator signal. In such embodiments, the central point of a radial spoke measured by each channel in a receiver coil array may be used as a motion signal. It may also be advantageous in some cases to incorporate into the analysis, alongside the center point, one or more other points from the trajectory. This may be advantageous in some cases because information from the MR signals for the other points could improve signal-to-noise ratio of the MR signals when analyzed for patient motion and/or diagnostic image quality. Such other points may include points that are adjacent in the trajectory to the center point in the trajectory or within a number of samples in the trajectory of the center point, within a threshold distance in k-space, or other points. For example, in a radial spoke, the other points could be in a row along a spoke. Motion signals determined from analysis of MR signals captured by one or more coils and from one or more k-space points may be combined using a variety of different computations, such as mean percentage change across channels and cross correlation between MR signal vectors, to create a quantitative metric of patient motion. As discussed elsewhere herein, a quantitative metric may then be compared to one or more thresholds and, based on whether any threshold is met and/or which threshold is met, a quantitative and/or qualitative estimate of diagnostic image quality may be generated. As mentioned above, in some embodiments in which different sequences or trajectories are being used, one or more thresholds may be determined for each such different sequence/trajectory. Having different thresholds may account for differences in MR signals that may be read out in the different sequences/trajectories and may also account for different impacts that patient motion may have on image quality between the different sequences/trajectories. In some cases, a patient motion during one sequence/trajectory may be large enough to inhibit generation of an MR image of sufficient diagnostic quality, but the same patient motion during another sequence/trajectory may have a lesser impact and an MR image of sufficient diagnostic quality may still be capturable.

In some embodiments in which a sequence or trajectory incorporates radial spokes, a center of mass analysis may be performed as part of analyzing MR signals to determine patient motion and/or image quality. Translational motion during an MRI scan may be determined, using MR signals for radial k-space spokes, through analysis of the center of mass (COM) of the projection moments of those multiple spokes. Multi-channel receive coil arrays may in some cases reduce accuracy of quantitative, translational COM motion estimates, as patient motion during the MRI scan may cause image content acquired by each coil to change over time due to the localized sensitivity profiles. This can modulate the MR signals acquired from imaging of the moving patient in a multiplicative fashion. The varying modulation of the object by multiple receive coil sensitivities, however, may make the COM estimates even more sensitive to patient motion within the coil array. Accordingly, multi-channel COM estimates could be analyzed in a manner similar to the discussion above (in connection with FIG. 2) and below (in connection with FIG. 3) to analysis of multi-channel FID navigator signals and/or central radial k-space signals. This analysis may generate a motion metric that could be compared to one or more thresholds to determine a quantitative and/or qualitative estimate of diagnostic image quality. For example, the multi-channel COM estimates, which give the center-of-mass of the object in x, y, and z, as viewed by each coil, could be combined using root-mean-square, and then averaged across all channels or across maximally changing channels (which may be the channels most sensitive to patient motion).

In some embodiments, FID navigator signals and central radial spoke data may also be combined with other calibration data acquired during an MRI scan and analyzed using a forward model of signal generation, which may generate six-degrees-of-freedom motion information. Such an approach is described in more detail below, following the discussion of FIG. 3. Such motion information may be usable for retrospective correction of acquired k-space data and for prospective steering of the acquisition. These motion signals may also be compared to one or more thresholds to determine a quantitative or qualitative estimate of diagnostic image quality. For example, calibrated motion signals may be combined into a root-mean-square motion score: score=\sqrt{δx²+δy²+δz²} where δx, δy and δz are the calibrated displacement in millimeters.

As mentioned above, embodiments are not limited to using any particular sequence or trajectory. While a detailed discussion has been provided of analyzing FID navigator signals or MR signals captured in a radial spoke trajectory, other sequences or trajectories may be used, including other trajectories that repeatedly sample the central k-space signal. In such a case, the center point of k-space and/or one or more other points adjacent in the trajectory may be analyzed to determine a measure of patient motion and/or diagnostic image quality. The discussion above of techniques for analyzing MR signals corresponding to central points from a radial spoke sequence/trajectory may be used in connection with analyzing central points from other sequences or trajectories.

### Example Embodiments

Described below are certain non-limiting examples of evaluating MR signals, determining diagnostic quality of an image that would result from an MRI scan, and evaluating prediction of diagnostic quality of such an image. These are intended to be illustrative of various ways in which techniques described herein may operate. It should be appreciated that embodiments are not limited to operating in accordance with these examples.

FIG. 3 is a flowchart describing an illustrative process 300 for evaluating prediction of diagnostic image quality using the MRI system. The process 300 includes four steps: qualitative assessment 302, combining measured FID navigator signals 304, determining an FID navigator motion score 306, and ROC analysis 308.

In some embodiments, at act 302, a radiologist may evaluate image data, resulting from one or more MRI scans, to determine an image grade based on the severity of motion artifacts and diagnostic image quality.

Concurrently or separately with the radiologist in act 302, a signal analysis facility being executed by a computing system may evaluate FID navigator signal data for the MRI scan(s) to determine an FID navigator motion score, indicative of the overall change in FID navigator signals over time. In particular, according to some embodiments, the computing system may combine measured FID navigator signals at act 304 to determine one or more global FID navigator metrics. Based on the global FID navigator metrics, the computing system may determine the FID navigator motion score at act 306.

In some embodiments, at act 308, the image grades and corresponding FID navigator motion scores may be used as input to a receiver operating characteristic (ROC) analysis. The ROC analysis may be performed to compare the sensitivity and specificity of each FID navigator-based metric (e.g., FID navigator motion scores, global FID navigator metrics) to detect differences between non-diagnostic images (Grades 1-2) and images with some diagnostic value (Grades 3-5), as well as between images with impaired diagnostic quality (Grades 1-3) and fully diagnostic images (Grades 4-5). In some embodiments, MR data (e.g., image data and/or FID navigator signal data) may be acquired prior to process 300 and used as input to act 302 and/or act 304. In some embodiments, this may include, for example, using an MRI system to acquire one or more MRI sequences in which an FID navigator module is embedded. For example, an FID navigator module may be embedded in the MRI sequence following each RF excitation pulse, prior to image readout.

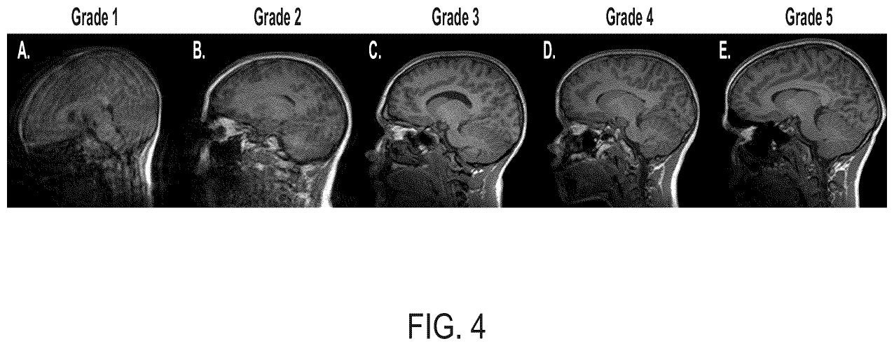

Returning to act 302, image data generated as a result of the MRI scans may be graded by a radiologist based on the severity of motion artifacts and diagnostic image quality. For example, images may be ranked using a five-point scale as follows: 1) severe motion artifact, non-diagnostic image without anatomic information (e.g., gross anatomic distortion such as hydrocephalus or a large tumor would be obscured); 2) severe motion artifact, non-diagnostic image with limited gross anatomic information (e.g., ventricular size, midline shift); 3) moderate motion artifact, diagnostic quality is compromised but some information is still obtained; 4) mild motion artifact, exam remains fully diagnostic; 5) no appreciable motion artifact. FIG. 4 is a series of example images acquired in pediatric patients, representative of each of the five image grades, as evaluated by a radiologist. In some embodiments, a subset of MRI scans may also be graded by a second radiologist to establish inter-rater reliability.

Returning to act 304, FID navigator signal data may be combined by the computing system to determine one or more global FID navigator metrics. As described herein, including with respect to act 202 of FIG. 2, FID navigator samples from all readouts in each echo train may be averaged to yield a single complex navigator signal per channel for a TR. To compress the multi-channel FID navigator signal data into a single global FID navigator motion metric per TR, individual coil measurements may be combined using any suitable algorithm. For example, Equations 1, 2, and/or 3 may be used to determine a single global FID navigator metric per TR.

Equation 1 may be used to determine the normalized mean absolute change in FID navigator signals over all channels (FIDₙₐᵥ_{_{Δ}}):

$${FIDnav}_{Δ} ( i ) = \frac{1}{{N}_{c}} {\sum }_{j = 1}^{{N}_{c}} \frac{\vert \vert {s}_{j} ( i ) \vert - \vert {s}_{j} ( i - 1 ) \vert \vert}{\vert {s}_{j} ( i - 1 ) \vert} \qquad Equation 1$$

where sⱼ(i) is the complex FID navigator signal measured from the jth coil at the ith time point (TR) and N_{c} is the number of channels in the coil array. In some embodiments, Equation 1 may be used to detect motion relative to the previous repetition, rather than an initial reference time point. This may be chosen because subjects generally do not return to their initial position following a motion event.

Equation 2 may be used to determine the normalized mean change in absolute FID navigator signals over channels (e.g., three channels) with maximal signal change at each time point (FIDₙₐᵥ_{_{Δmax}}):

$${FIDnav}_{Δ max} ( i ) = \frac{1}{3} {\sum }_{j = 1}^{3} \vert \frac{\vert {s}_{j} ( i ) \vert - \vert {s}_{j} ( i - 1 ) \vert}{\vert {s}_{j} ( i - 1 ) \vert} \vert \qquad Equation 2$$

This metric may increase sensitivity to fast motion by only averaging data from channels exhibiting maximum signal change. Averaging over the most changing three channels may reduce the effect of random signal fluctuations, while increasing motion-induced signal changes.

Equation 3 may be used to determine the cross-correlation coefficient (CCC) between absolute FID navigator signal projection vectors (FIDnav_{CCC}):

$${FIDnav}_{CCC} ( i ) = 1 - \frac{{\sum }_{j = 1}^{{N}_{c}} [ ( {s}_{j} ( i ) - \overline{{s}_{j} ( ι )} ) ( {s}_{j} ( i - 1 ) - \overline{{s}_{j} ( ι - 1 )} ) ]}{\sqrt{\frac{1}{{N}_{c} - 1} {\sum }_{j = 1}^{{N}_{c}} {( {s}_{j} ( i ) - \overline{{s}_{j} ( ι )} )}^{2}} \sqrt{\frac{1}{{N}_{c} - 1} {\sum }_{j = 1}^{{N}_{c}} {( {s}_{j} ( i - 1 ) - \overline{{s}_{j} ( ι - 1 )} )}^{2}}} \qquad Equation 3$$

In some embodiments, a change in CCC relative to the previous TR may imply that the load distribution of the coil elements has changed, indicating a motion event.

At act 306, to evaluate the ability of FID navigator signals to predict motion affecting diagnostic image quality, the computing system may further compress the global FID navigator metrics into single measures of the total impact of motion occurring during the MRI scan. For example, Equations 4 and/or 5 may be used to determine an FID navigator motion score.

Equation 4 may be used to determine an integrated FID navigator motion score per second (FIDnav):

$$FIDnav = \frac{1}{n \cdot TR} {\sum }_{i = 1}^{n - 1} {FIDnav}_{i} \qquad Equation 4$$

In some embodiments, FIDnav represents the numerical integration of the total FID navigator motion score per second, where n is the total number of k-space lines in the acquisition.

Equation 5 may be used to determine a partition-weighted FID navigator motion score (wFIDnav):

$$wFIDnav = \frac{1}{n \cdot TR} {\sum }_{i = 1}^{n} {w}_{i} \cdot {FIDnav}_{i} \qquad Equation 5$$

where wᵢ represents the weighting associated with the ith k-space plane. This metric may reflect the fact that k-space energy and hence the impact of motion occurring at each encoding step may be non-uniformly distributed along the phase-encoding direction. For the experiment described herein, weightings were calculated from the norm of each acquired k-space plane, averaged across the eight co-operative adult subjects scanned with the same sequence parameters.

In some embodiments, Spearman rank correlation may be evaluated to determine whether the aggregate FID navigator-based motion metrics (e.g., the FID navigator motion scores determined at act 306) correlate with radiologic evaluation grade determined at act 302. P-values may be calculated to determine if group-wise differences are statistically significant.

In some embodiments, a receiver operating characteristic (ROC) analysis may be performed to compare the sensitivity and specificity of each FID navigator-based metric (e.g., determined at act 306) to detect differences between non-diagnostic images (Grades 1-2) and images with some diagnostic value (Grades 3-5), as well as between images with impaired diagnostic quality (Grades 1-3) and fully diagnostic images (Grades 4-5). Sensitivity (SE) and specificity (SP) may be defined from the ratio of true and false positives (TP, FP) and true and false negatives (TN, FN) as follows:

$$SE = \frac{TP}{TP + FN} \qquad Equation 6$$

$$SP = \frac{TN}{TN + FP} \qquad Equation 7$$

The threshold for detection of image quality degradation may in some embodiments be determined using Youden's index, which is a threshold that maximizes SE+SP. In some embodiments, as described herein including with respect to act 204 of process 200, an FID navigator motion score may be compared to such a threshold to determine diagnostic quality of an image that would result from an MRI scan. For example, if the FID navigator motion score exceeds the threshold, this may indicate that the completed MRI scan would result in an image that is non-diagnostic.

In some embodiments, the techniques described herein may be used to save time. For example, potential time savings may be realized by terminating the acquisition of motion-corrupted MRI scans at the point when the FID navigator-based metrics (e.g., FID navigator motion score) exceeds the threshold (e.g., will result in a non-diagnostic image). In some embodiments, an analysis may be conducted to quantify the potential time savings. For this analysis, it may be assumed that all images ranked in Grades 1-2 would be reacquired following completion of the non-diagnostic MRI scan, giving a total acquisition time of 2TA, whilst MRI scans with acceptable or good diagnostic quality (Grades 3-5) would not be repeated. Thus, for any MRI scan of non-diagnostic image quality, if the integrated motion metric exceeds the predefined threshold value (computed from ROC analysis), the fraction of the time remaining (FR) may be recorded, as this represents the time saved in acquiring non-diagnostic image data. For any MRI scan of diagnostic quality, if the integrated motion metric exceeds the threshold value, the fraction acquired (FA) may be recorded, as this represents time wasted unnecessarily restarting the MRI scan. The total time savings may be computed across all subjects and expressed as a percentage of the total MRI scan time:

$${T}_{Savings} ( \% ) = 100 \cdot ⌈ {\sum }_{i = 1}^{nND} ( 1 - \frac{{FR}_{i}}{2} ) - {\sum }_{i = 1}^{nD} ( 1 + {FA}_{i} ) ⌉ \qquad Equation 8$$

where nND and nD represent the number of subjects with non-diagnostic and diagnostic MRI scans, respectively.

As mentioned above, embodiments are not limited to analyzing MR signals that are FID navigator signals. Other MR signals captured during an MRI scan may also be analyzed to determine patient motion and/or image quality. An example of a technique for analyzing MR signals that are not FID navigator signals is provided below.

As a point of comparison for the discussion below of analyzing central k-space signals, an FID navigator signal from coil/and acquisition n may be expressed as:

$${y}_{j , n} ( τ ) = \int {s}_{j , n} ( r ) \cdot ρ ( r , τ ) \cdot exp ( i γτδ {B}_{0 , n} ( r ) ) dr \qquad [ 1 ]$$

where s_{j,n}(r) is the complex coil sensitivity profile at position r for object pose n, ρ(r, τ) is the effective proton density at the FID navigator sampling time τ; γ is the gyromagnetic ratio, and δB_{0,n}(r) describes off-resonant effects, including eddy currents and motion-induced changes in the magnetic field.

The central k-space signal from coil j and spoke n may be expressed as:

$${y}_{j , n} ( TE ) = \int {s}_{j , n} ( r ) \cdot ρ ( r , TE ) \cdot exp ( i γ TE δ {B}_{0 , n} ( r ) ) dr \qquad [ 1 ]$$

where s_{j,n}(r) is the complex coil sensitivity profile at position r for object pose n; ρ(r, TE) is the effective proton density at echo time TE; γ is the gyromagnetic ratio, and δB_{0,n}(r) describes off-resonant effects, including non-isotropic gradient delays and gradient moment imbalances due to eddy currents, as well as motion-induced changes in the magnetic field.

The central point of k-space (e.g., of a radial spoke, a spiral, or other trajectory) may be viewable as a self-encoded FID navigator, subject to off-resonance effects, which cause the trajectory to deviate from the intended path. These off-resonance effects may be effectively modelled in some cases by low-spatial-order spherical harmonic (SH) expansion. Thus, if the subject moves and/or if there is a change in off-resonance effects, this will induce a change in the measured central k-space signal from each coil in the array.

To calibrate a motion measurement model, coil sensitivity information sⱼ(r) may be obtained from the pre-scan normalization data acquired by the MRI scanner prior to a scan, which in some cases may be automatically captured by the MRI scanner (if so configured by an operator of the machine, vendor, or other) on the surface and body coils. The coil sensitivity profiles may be computed by complex division of the acquired low-resolution surface and body coil images and extrapolated over the imaging field-of-view by fitting a thin-plate spine function to sampled data points. Relative motion between the coils and the underlying object may be simulated in six degrees of freedom by re-evaluating this mathematical model of the coil profiles in the transformed co-ordinate system. A low-resolution image may be reconstructed from the first N pseudo-randomly ordered signals (e.g., radial spokes) or from a separate (e.g., Cartesian) scan to provide an estimate of the effective proton density ρ(r, TE). Changes in SH coefficients up to second order (described by 9 coefficients) in the head frame of reference can also be simulated. Together these can be used to create a forward model:

$$Y = Au \qquad [ 2 ]$$

where Y is a vector of the complex central k-space signals from N_{c} receiver coils, A is the calibration matrix of dimensions N_{c}×(N_{P}+1), where Nₚ is the number of unknowns to be estimated at each time point; and u=[1, d₁, d₂, d₃, ϕ₁, ϕ₂, ϕ₃, b₀ . . . bₙ]^{T} where dₙ, ϕₙ and bₙ are the unknown translation, rotation and B₀ field coefficients, respectively. Such a calibrated motion measurement model may be used to determine patient/subject motion and/or to estimate quality of an image that has or may result from an MRI scan, as discussed above.

### Example Experiments

Experiments were undertaken to investigate the ability of FID navigator motion detection techniques to prospectively predict diagnostic utility, according to the example process described herein including with respect to FIG. 3. The sensitivity and specificity of various FID navigator detection algorithms were evaluated relative to radiologic evaluation of image quality in a pediatric patient cohort.

A total of 102 pediatric patients (49 female) were scanned between October 2017 and August 2019. Patients' ages ranged from 0 to 18 years, with median age 14 years. Eight adult patients (age 19-35) were also scanned. Patients were scanned for a range of clinical indications at a hospital's outpatient facility. The retrospective use of patient data was approved by the local Institutional Review Board (IRB) and the need for informed consent was waived.

Patients were scanned without sedation at 3 T (MAGNETOM Trio, A Tim System; Siemens, Erlangen, Germany) using the vendor-supplied 32-channel head coil. A T1-weighted MPRAGE sequence was acquired for each patient with the following MRI scan parameters: TR 1540 ms, TI 800 ms, TE 2.47 ms, flip angle 9°, receiver bandwidth 200 Hz/pix, FOV 220×220×152 mm, resolution 0.9 mm isotropic, GRAPPA acceleration factor 2, total acquisition time 4.2 min. An FID navigator module (duration 0.2 ms) was embedded in the sequence following each RF excitation pulse, prior to image readout. A Cartesian sampling trajectory was used with the center of the phase encoding direction acquired halfway through the scan. Acquisitions were non-selective and acquired in the sagittal plane. Sequence parameters were chosen to closely match those typically used in clinical studies. N=26 MRI scans were acquired with water excitation as per the clinical protocol. N=12 MRI scans were acquired post-contrast. Foam padding was used within the head coil to restrict motion, as is done in clinical practice. No specific instructions were given to subjects, other than to remain as still as possible for the duration of the MRI scan.

Image data resulting from the MRI scans was assessed by two experienced radiologists to determine image quality scores. The presence of severe motion artifacts meant that 12% of all MRI scans were non-diagnostic (Grades 1-2). A further 12% had compromised diagnostic value due to motion (Grade 3). There was excellent agreement between the two raters' evaluation of image quality (Cohen's weighted κ=0.86, 95% CI [0.68-1.0], P<0.001). All disagreements between raters occurred for MRI scans with quality ratings in adjacent categories. As described herein above, representative images corresponding to each grade are shown in FIG. 4.

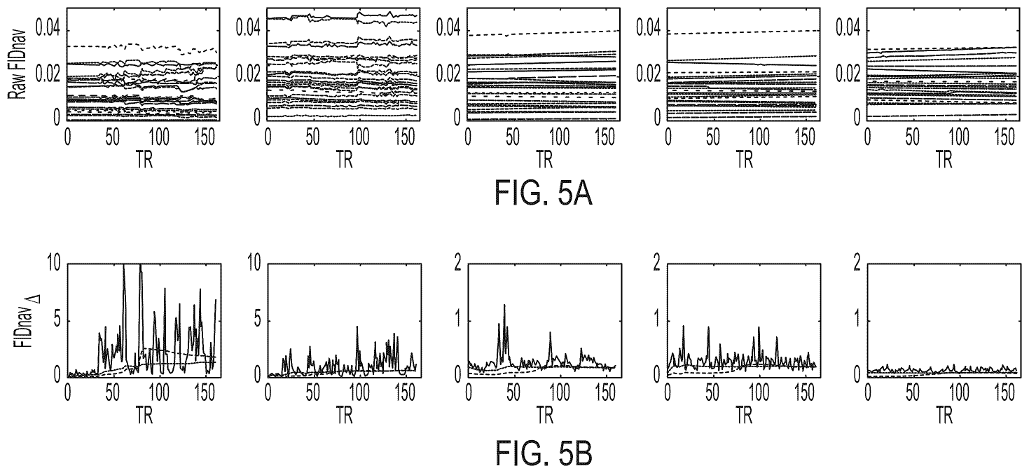

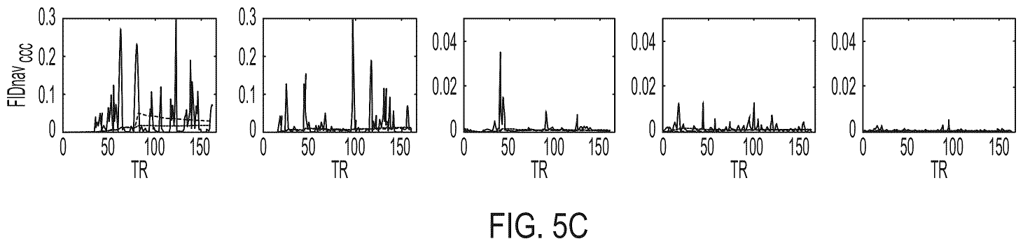

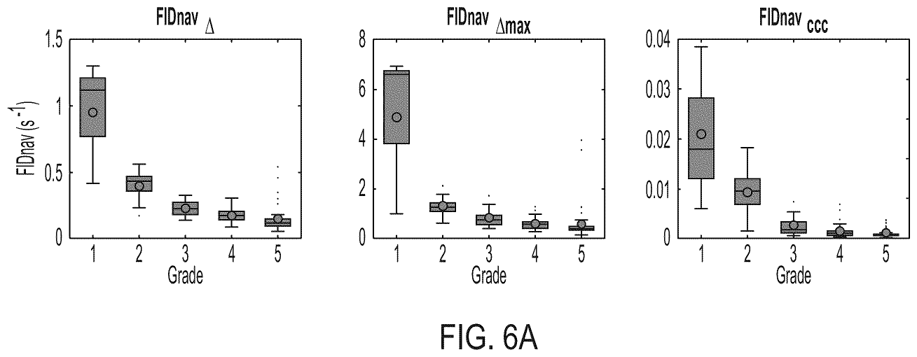

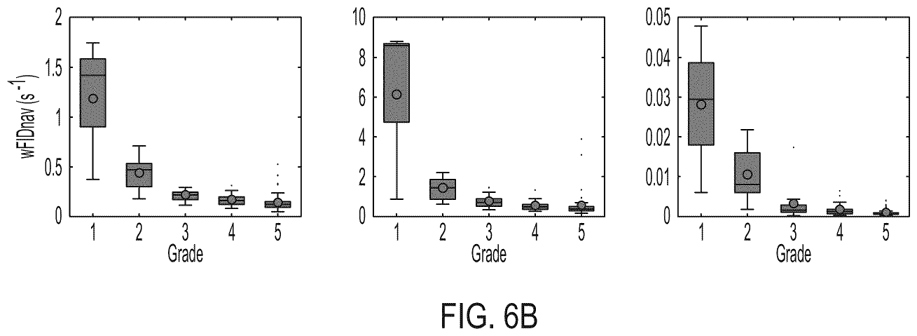

FIG. 5A shows the multi-channel FID navigator signal traces corresponding to the images from Grades 1-5 displayed in FIG. 4. The temporal resolution of FID navigator motion measurement is determined by the TR of the sequence, which was 1.54 s in this experiment. The derived metrics FIDnav_{Δ} and FIDnav_{CCC} are shown in FIGS. 5B and 5C, respectively, alongside the integrated (red) and partition-weighted (purple) integrated motion scores for each subject. The relationship between aggregate FID navigator motion scores and radiologic evaluation of image quality is summarized in FIG. 6. The mean and standard deviation of FID navigator metrics corresponding to each motion grade is displayed in Table 1. All metrics were significantly correlated with image grade (Spearman rank correlation coefficient 0.57-0.61; P<0.001). Weighting the motion metrics by an estimate of the k-space signal at each time point did not improve the correlation with image quality.

TABLE 1
Summary integrated FID navigator statistics (mean ± standard
deviation) for each image grade derived from each FID navigator metric.

| | FIDnav (s⁻¹) | | | wFIDnav (s⁻¹) | | |
|---|---|---|---|---|---|---|
| | FIDnav_{Δ} | FIDnav_{Δmax} | FIDnav_{CCC}* | FIDnav_{Δ} | FIDnav_{Δmax} | FIDnav_{CCC}* |
| Grade 1 | 0.95 ± 0.47 | 4.85 ± 3.33 | 2.09 ± 1.65 | 1.18 ± 0.71 | 6.10 ± 4.51 | 2.78 ± 2.10 |
| (n = 3) | | | | | | |
| Grade 2 | 0.39 ± 0.12 | 1.29 ± 0.48 | 0.92 ± 0.50 | 0.43 ± 0.17 | 1.42 ± 0.59 | 1.04 ± 0.70 |
| (n = 9) | | | | | | |
| Grade 3 | 0.22 ± 0.06 | 0.82 ± 0.40 | 0.26 ± 0.21 | 0.21 ± 0.06 | 0.75 ± 0.33 | 0.31 ± 0.46 |
| (n = 12) | | | | | | |
| Grade 4 | 0.17 ± 0.06 | 0.56 ± 0.25 | 0.16 ± 0.16 | 0.16 ± 0.06 | 0.54 ± 0.24 | 0.16 ± 0.15 |
| (n = 29) | | | | | | |
| Grade 5 | 0.14 ± 0.09 | 0.56 ± 0.70 | 0.10 ± 0.09 | 0.14 ± 0.09 | 0.53 ± 0.66 | 0.10 ± 0.09 |
| (n = 49) | | | | | | |
| Spearman | 0.61 | 0.57 | 0.57 | 0.60 | 0.55 | 0.55 |
| Correlation | | | | | | |

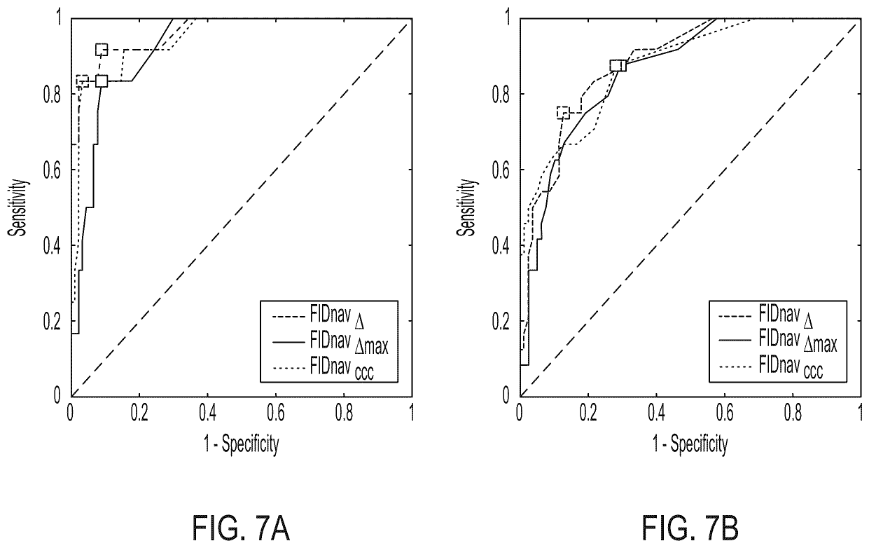

The results of the ROC analysis for all metrics of this example are summarized in Table 2. Computing the cross-correlation coefficient between FID navigator signal vectors (FIDnav_{CCC}) achieved the highest sensitivity and specificity (>90%) for detecting non-diagnostic images (FIGS. 7A-B), outperforming computing the percentage signal change across all or maximally changing channels. A sensitivity of 92% for FIDnav_{CCC} means that 92% of non-diagnostic images were correctly identified, while a specificity of 91% means that 9% of diagnostic images were incorrectly flagged as non-diagnostic. The FID navigator-based metrics were less sensitive in detecting images with impaired diagnostic quality, with sensitivity 88% and specificity 72% for FIDnav_{CCC}.

TABLE 2
Receiver operating characteristic (ROC) analysis area under
the curve, sensitivity and specificity, and optimal threshold
for each integrated FID navigator motion score.

| | Grades 1-2 vs 3-5 | | | | Grades 1-3 vs 4-5 | | | |
|---|---|---|---|---|---|---|---|---|
| Integrated Motion | | SE² | SP³ | | | SE | SP | |
| Score | AUC¹ | (%) | (%) | Th⁴ | AUC | (%) | (%) | Th |
| FIDnav_{Δ} | 0.94 | 0.83 | 0.97 | 0.20 | 0.87 | 0.75 | 0.87 | 0.13 |
| FIDnav_{Δmax} | 0.91 | 0.83 | 0.91 | 0.59 | 0.85 | 0.88 | 0.71 | 0.31 |
| FIDnav_{CCC} | 0.95 | 0.92 | 0.91 | 2.0e−3 | 0.86 | 0.88 | 0.72 | 7.9e−4 |

¹AUC: area under the operating curve;
²SE: sensitivity;
³SP: specificity;
⁴Th: optimal threshold

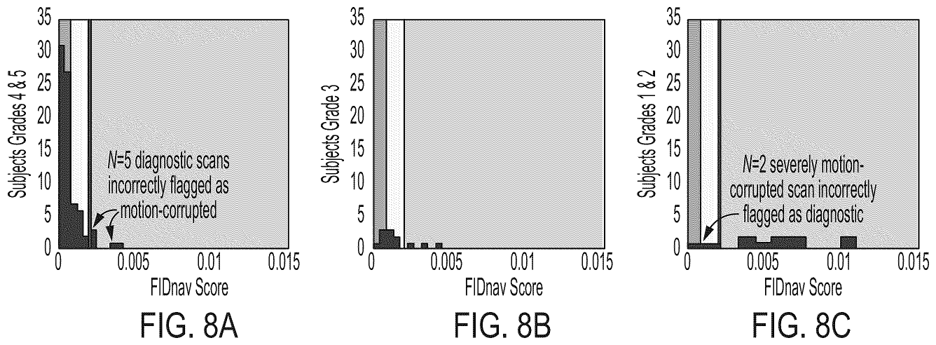

The integrated cross-correlation between FID navigator signal vectors (FIDnav_{CCC}) was used in the potential time savings analysis as this metric was shown to have the highest overall detection power. Histograms of this integrated FID navigator motion score are shown in FIGS. 8A-C for subjects in Grades 1-2 (non-diagnostic), Grade 3 (some diagnostic value), and Grades 4-5 (fully-diagnostic), respectively, alongside the optimal thresholds for detecting motion-corrupted MRI scans. Applying the optimal threshold to detect non-diagnostic images yielded total time savings of 8% across all MRI scans (N=102); less than 2% of the overall imaging time was ‘wasted’ reacquiring diagnostic quality images. Applying the more stringent FID navigator threshold (to detect impaired diagnostic image quality) resulted in 9% time savings. With this more stringent threshold, more time was saved in acquiring non-diagnostic quality images but the rate of false-positives also increased.

The aim of the experiments was to investigate the ability of FID navigator-based motion metrics to determine clinically relevant levels of motion in pediatric patients scanned in a realistic clinical setting. The results show that integrated motion metrics from FID navigators embedded in a structural MPRAGE sequence are correlated with expert radiologic evaluation of image quality. Integrating the cross-correlation between FID navigator signal vectors (FIDnavCCC) had the highest power for detecting non-diagnostic images. Applying a cut-off threshold to this integrated FID navigator metric demonstrated that substantial time and cost savings could be realized by using this metric inform termination of non-diagnostic acquisitions prior to completion.

In the experiments, MRI scans ranked as Grade 4 and 5 were judged to be fully diagnostic. MRI scans ranked as Grade 3 were corrupted by motion to a degree that they still retained some diagnostic value. The need to repeat these MRI scans may depend on both the clinical context and quality of other images in the series. MRI scans ranked as Grades 1-2 were severely corrupted by motion and deemed to have no clinical value. The results of the current study indicate that FID navigator signals may be useful in flagging severely motion-corrupted, non-diagnostic images, which would need to be repeated regardless of clinical indication. FID navigator signals had high sensitivity and moderate specificity for detecting more subtle motion artifacts present in the Grade 3 images.

Computing the integrated cross-correlation between multi-channel FID navigator signals has high sensitivity and specificity for detecting clinically relevant pediatric head motion in structural brain images. Motion detection with FID navigator signals has potential to increase MRI scan efficiency and patient throughput both by minimizing time acquiring non-diagnostic information and by reducing the need for sedation.

### Illustrative Applications

MR signal-based motion scores may be used to provide feedback to the operator (e.g., the imaging technologist) to identify non-diagnostic MRI scans before the acquisition is complete or to trigger a prospective correction strategy to steer the FOV after detection of a motion event. Real-time or otherwise contemporaneous feedback of motion information could also be given directly to the subject, which may be effective in increasing subject compliance. In addition to directly reducing the time spent acquiring motion-corrupted data, metrics for real-time motion detection may also yield other practical time savings by avoiding delays that arise from waiting to confirm with the radiologist that images are of sufficient quality before the patient is taken off the MRI scan table, as well as minimizing the patient call-back rate due to non-diagnostic imaging.

MR signal motion information may also be used retrospectively to identify motion-corrupted data to improve the reconstruction. The inclusion of low-impact FID navigator tracking information in a wide range of MRI scans may be used to control and quantify the confounding effects of motion in quantitative group-wise analysis. MR signal-based motion metrics may be included as a nuisance regressor or used to match levels of motion between groups to increase the validity of cross-sectional and longitudinal comparison studies.

The use of MR signals may be an attractive approach for pediatric head motion monitoring as they can be acquired with high temporal resolution in virtually any sequence, with low time penalty, and do not require any additional hardware or fiducial markers. Combining MR signal data from multiple channels into a single metric enables real-time motion monitoring and the use of an empirical threshold to detect different levels of motion and/or diagnostic quality. This may be used to reduce motion sensitivity and improve scan efficiency, saving time and money, and reducing discomfort to the patient.

### Example Processing Techniques

Techniques operating according to the principles described herein may be implemented in any suitable manner. Included in the discussion above are a series of flow charts showing the steps and acts of various processes that analyze MR data to evaluate an image that would result from an MRI scan. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way: all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application, for example as a software program application such as a signal analysis facility.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

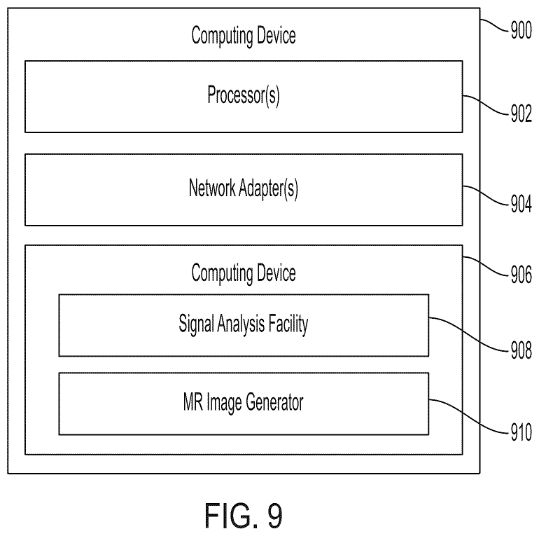

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 906 of FIG. 9 described below (i.e., as a portion of a computing device 900) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 9, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 9 illustrates one exemplary implementation of a computing device in the form of a computing device 900 that may be used in a system implementing techniques described herein, although others are possible. It should be appreciated that FIG. 9 is intended neither to be a depiction of necessary components for a computing device to operate as a device for evaluating an image that would result from an MRI scan in accordance with the principles described herein, nor a comprehensive depiction.

Computing device 900 may comprise at least one processor 902 (e.g, a computer hardware processor), a network adapter 904, and computer-readable storage media 906. Computing device 900 may be, for example, a desktop or laptop personal computer, a personal digital assistant (PDA), a smart mobile phone, or any other suitable computing device. Network adapter 904 may be any suitable hardware and/or software to enable the computing device 900 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 906 may be adapted to store data to be processed and/or instructions to be executed by processor 902. Processor 902 enables processing of data and execution of instructions. The data and instructions may be stored on the computer-readable storage media 906.

The data and instructions stored on computer-readable storage media 906 may comprise computer-executable instructions implementing techniques which operate according to the principles described herein. In the example of FIG. 9, computer-readable storage media 906 stores computer-executable instructions implementing various facilities and storing various information as described above. Computer-readable storage media 906 may store signal analysis facility 908 configured to evaluate an image that would result from an MRI scan. Media 906 may also store an MR image generator 910 to generate one or more images from signals captured during an MRI scan.

While not illustrated in FIG. 9, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second.” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1. A method for evaluating at least one image that would result from a magnetic resonance image (MRI) scan, the method comprising:
evaluating magnetic resonance (MR) signals captured during the MRI scan;
determining a diagnostic quality of the at least one image that would result from the MRI scan, based at least in part on a result of evaluating the MR signals; and
outputting an indication of the diagnostic quality of the at least one image.

2. The method of claim 1, wherein determining the diagnostic quality of the at least one image that would result from the MRI scan comprises:
comparing the result of the evaluating of the MR signals to a threshold; and
determining that the diagnostic quality of the at least one image that would result from the MRI scan is non-diagnostic when the result of evaluating the MR signals exceeds the threshold.

3. The method of claim 1, wherein evaluating the MR signals captured during the MRI scan comprises determining a change in the MR signals over time.

4. The method of claim 3, wherein determining the diagnostic quality of the at least one image that would result from the MRI scan comprises:
comparing the change in the MR signals to a threshold; and
determining that the diagnostic quality of the at least one image that would result from the MRI scan is non-diagnostic when the change in the MR signals exceeds the threshold.

5. The method of claim 1, wherein:
the MR signals comprise free-induction decay (FID) navigator signals captured by two or more receiver coils during the MRI scan; and
evaluating the MR signals captured during the MRI scan comprises combining the MR signals captured by the two or more receiver coils to determine at least one first metric.

6. The method of claim 5, wherein combining the MR signals comprises combining MR signals captured for a channel, for a time, and/or for the MRI scan.

7. The method of claim 5, wherein combining the MR signals captured by the two or more receiver coils comprises determining a normalized mean absolute change in the MR signals and/or a cross correlation coefficient between projection vectors of the MR signals.

8. The method of claim 5, wherein:
evaluating MR signals captured during the MRI scan comprises evaluating FID navigator signals captured during the MRI scan; and
evaluating the FID navigator signals captured during the MRI scan further comprises integrating the at least one first metric with respect to a number of k-space lines to determine at least one second metric.

9. The method of claim 8, wherein determining the diagnostic quality of the at least one image that would result from the MRI scan comprises:
comparing the at least one second metric to a threshold; and
determining that the at least one image that would result from the MRI scan is non-diagnostic when the at least one second metric exceeds the threshold.

10. The method of claim 1, wherein determining the diagnostic quality of the at least one image that would result from the MRI scan comprises determining the diagnostic quality during the MRI scan, prior to image reconstruction.

11. The method of claim 1, wherein outputting the indication of the diagnostic quality of the at least one image that would result from the MRI scan comprises outputting the indication of the diagnostic quality during the MRI scan, prior to image reconstruction.

12. The method of claim 1, wherein outputting the indication of the diagnostic quality comprises recommending intervention when the diagnostic quality is determined to be non-diagnostic.

13. (canceled)

14. The method of claim 1, wherein outputting the indication of the diagnostic quality comprises displaying data through a user interface of an MRI system.

15. (canceled)

16. The method of claim 1, wherein the MR signals comprise FID navigator signals captured during an acquisition of one or more MRI sequences, and the one or more MRI sequences comprise embedded FID navigator modules following RF excitation pulses.

17. The method of claim 1, wherein evaluating the MR signals captured during the MRI scan comprises evaluating MR signals corresponding to a center of k-space captured during an MRI scan in which the center of k-space was repeatedly sampled.

18. The method of claim 17, wherein evaluating MR signals corresponding to the center of k-space captured during an MRI scan in which the center of k-space was repeatedly sampled comprises evaluating MR signals corresponding to the center of k-space and that were captured without gradient encoding.

19.-21. (canceled)

22. A magnetic resonance imaging (MRI) system configured to evaluate a magnetic resonance image (MRI) scan, comprising:
an MRI scanner;
at least one processor; and
at least one non-transitory computer-readable storage medium storing executable instructions that, when executed by the at least one processor, cause the at least one computer hardware processor to perform a method for evaluating at least one image that would result from a magnetic resonance image (MRI) scan, the method comprising:
evaluating magnetic resonance (MR) signals captured during the MRI scan;
determining a diagnostic quality of the at least one image that would result from the MRI scan, based at least in part on a result of evaluating the MR signals; and
outputting an indication of the diagnostic quality of the at least one image.

23. A system configured to evaluate a magnetic resonance image (MRI) scan, comprising:
at least one processor; and
at least one non-transitory computer-readable storage medium storing processor executable instructions that, when executed by the at least one processor, cause the at least one processor to perform a method for evaluating a magnetic resonance image (MRI) scan, the method comprising:
estimating motion of a subject during an MRI scan based on measured magnetic resonance (MR) signals captured during the MRI scan;
determining a diagnostic quality of the MRI scan based on the estimated motion; and
outputting an indication of the diagnostic quality of the MRI scan.

24. The system of claim 23, wherein estimating the motion based on the measured MR signals captured during the MRI scan comprises estimating the motion based on measured free-induction decay (FID) navigator signals captured during the MRI scan.

25. The system of claim 23, wherein estimating the motion based on the measured MR signals captured during the MRI scan comprises estimating the motion based on measured MR signals corresponding to a center of k-space captured during an MRI scan in which the center of k-space was repeatedly sampled.

26.-30. (canceled)
